(19) United States
(12) Patent Application Publication
(10) Pub. No.: US 2017/0312209 A1
(43) Pub. Date: Nov. 2, 2017

(54) KERATINOUS FIBER CARE PRODUCT AND METHOD

(71) Applicant: Henkel AG & Co. KGaA, Duesseldorf (DE)

(72) Inventors: Volker Scheunemann, Lueneburg (DE); Thorsten Knappe, Schenefeld (DE); Erik Schulze zur Wiesche, Hamburg (DE)

(73) Assignee: Henkel AG & Co. KGaA, Duesseldorf (DE)

(21) Appl. No.: 15/531,736

(22) PCT Filed: Nov. 2, 2015
(86) PCT No.: PCT/EP2015/075397

§ 371 Date: May 31, 2017

(30) Foreign Application Priority Data
Dec. 10, 2014 (DE) 10 2014 225 425.6

(51) Int. Cl. A61K 8/64; A61K 8/92; A61K 8/894; A61K 8/34; A61K 8/898; B05B 17/04; A61K 8/891

(52) U.S. Cl. CPC A61K 8/64; B05B 17/04; A61K 8/92; A61K 8/891; A61K 8/345; A61K 8/898; A61K 8/894; A61K 2800/5426; A61K 2800/87

(57) ABSTRACT

Cosmetic products and methods for the care of keratin-containing fibers using the cosmetic products are provided herein. In an embodiment, a cosmetic product includes a) a cosmetic preparation and b) a device for the flash evaporation of the cosmetic preparation a). The a cosmetic preparation a) includes, based on its total weight, a1) from about 70 to about 99% by weight of a polar solvent and a2) at least about 0.1% by weight of care components.

### CROSS-REFERENCE TO RELATED APPLICATION

This application is a U.S. National-Stage entry under 35 U.S.C. §371 based on International Application No. PCT/EP2015/075397, filed Nov. 2, 2015, which was published under PCT Article 21(2) and which claims priority to German Application No. 10 2014 225 425.6, filed Dec. 10, 2014, which are all hereby incorporated in their entirety by reference.

### TECHNICAL FIELD

The application relates to the technical field of the care of keratin-containing fibers, especially human hair. The subject matter of the application are specific cosmetic formulations for hair, which are suitable for application on keratin-containing fibers by means of a flash evaporation process. Moreover, the use of these cosmetic formulations for hair in devices for flash evaporation and methods for the temporary deformation of keratin-containing fibers are the subject matter of the present application.

### BACKGROUND

An attractively appearing hairstyle is generally regarded at the present time as an indispensable part of a groomed appearance. In order to realize such an appealing hairstyle, the hair is subjected to cosmetic treatment methods ranging from cleaning by means of a shampoo to permanent shaping by means of chemical/thermal processes or permanent oxidative coloring. Many of these cosmetics method are connected with an intervention into the structure of hair and, depending on the frequency and intensity of the treatment; these methods are suitable for damaging the hair.

Many hair care materials are available for preventing such damage or eliminating it once it has already occurred. For the application of these hair care compositions, the spray application of appropriate cosmetic preparations is of great importance, the preparations generally being applied as pump sprays or aerosol sprays. For this purpose, the cosmetic preparations are packaged in a dispensing device, from which they are sprayed either by means of the mechanical action of a force or with the help of a propellant and a valve. Both methods have obvious disadvantages. Whereas pump sprays generally are not suitable for a long-lasting uniform spray applications of cosmetic preparations for the hair, aerosol sprays are based on the use of propellants or propellant gases which, on the one hand, do not develop a cosmetic effect and, on the other, can pose a risk to the consumer when handled improperly.

Against this background, there is a need for alternative routes for atomizing cosmetic preparations for the hair. Flash evaporation has proven to be such an alternative spraying method. For this method, which is described, for example, in the international patent application WO 2001/83071 A1 (Henkel), a liquid or pasty solvent-containing composition is heated in a closed space to a temperature, which is above the boiling point of the solvent, as a result of which pressure is produced in the composition. Upon relieving (throttling) the pressure, the liquid vaporizes and can subsequently be atomized, for example, by means of a suitable nozzle.

Even though flash evaporation is suitable, in principle, for the spray application of cosmetic preparations for the hair, at the same time, not every cosmetic preparation for the hair can be atomized by a flash evaporation method. This is due, on the one hand, to the heating of the cosmetic preparation, which is necessary for the flash evaporation, and, on the other, to the specific properties of the spray mist produced by the flash evaporation, for example, the droplet size and droplet density in the spray mist produced. It was therefore an object of the present disclosure to provide specific cosmetic preparations for hair for taking care of keratin-containing fibers, which, owing to their chemical and physical properties, are suitable for a purpose-directed spray application by means of a device for flash evaporation. Furthermore, the preparations should be suitable for realizing a high care effect and preferably a high degree of holding the hair in place, especially of doing so for a long time, and a high volume effect after an application by means of a flash method. It has turned out that, from the large number of known, effective cosmetic preparations for the hair, especially preparations with a high solvent content and based on care components are suitable for solving this problem.

### BRIEF SUMMARY

Cosmetic products and methods for the care of keratin-containing fibers using the cosmetic products are provided herein. In an embodiment, a cosmetic product includes a) a cosmetic preparation and b) a device for the flash evaporation of the cosmetic preparation a). The a cosmetic preparation a) includes, based on its total weight, a1) from about 70 to about 99% by weight of a polar solvent and a2) at least about 0.1% by weight of care components.

In another embodiment, a method for the care of keratin-containing fibers includes acting upon the keratin-containing fibers with a device for flash evaporation of a cosmetic preparation a). The cosmetic preparation a) includes, based on its total weight, a1) from about 70 to about 99% by weight of a polar solvent and a2) at least about 0.1% by weight of care components.

In another embodiment, a cosmetic product includes a) a cosmetic preparation a) that includes, based on its total weight, a1) from about 70 to about 99% by weight of a polar solvent and a2) from about 1.0 to about 10% by weight of care components. The care components a2) are selected from the group of i) cationic care components, ii) silicones, iii) polyols, iv) protein hydrolysates, v) waxes, or combinations thereof. The cosmetic product further includes b) a device for the flash evaporation of the cosmetic preparation a). The device b) includes a container b1) and a heater b2). The device b) is configured in such a way that the cosmetic preparation a) can be accommodated in the interior of the container b 1). The device b) is also configured in such a way that the interior of the container b1), which is at least partially filled with the cosmetic preparation a), can be closed off. The device b) is also configured in such a way that the cosmetic preparation a) in the closed interior of the container b1) can be heated with the heater b2) while the pressure increases. The device b) is also configured in such a way that the pressure on the heated cosmetic preparation a) from the interior of the container b1) can be lowered by being relieved into the surroundings.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first object of the present disclosure accordingly is a cosmetic product, comprising
a) a cosmetic preparation containing, based on its total weight,
   a1) from about 70 to about 99% by weight of a polar solvent,
   a2) at least about 0.1% by weight of care component;
b) a device for the flash evaporation of the cosmetic preparation a).

The cosmetic preparation a) is liquid. The cosmetic preparation a) may also be present as a solution or dispersion, for example, as an emulsion or suspension. Preferred cosmetic preparations a) are present in the form of a solution or a suspension.

As a first significant component, the inventive cosmetic preparation contains about 88% by weight of a polar solvent a1). Preferred cosmetic products are exemplified in that the proportion by weight of the polar solvent a1) amounts to from about 80 to about 99% by weight, preferably from about 90 to about 98% by weight and particularly from about 92 to about 97% by weight of the total weight of the cosmetic preparation a). Appropriate materials are distinguished by a good cosmetic effect and, at the same time, by good applicability.

In order to improve the application properties of the inventive cosmetic preparations while, at the same time, minimizing the thermal strain on any active ingredients or additives in the course of the flash evaporation method, it has proven to be advantageous to use polar solvents a1), which have a boiling point (20° C., 1013 mbar) of between about 50° and about 110° C. and preferably of between about 70° and about 105° C. Ethanol and water have proven to be particularly suitable in this connection and, for this reason, are preferred as polar solvents a1).

The following are particularly preferred polar solvents a1) or solvent systems:
polar solvents a1), for which the ratio of water to the total weight of the polar solvent a1) is more than about 80% by weight, preferably more than about 85% by weight and particularly more than about 90% by weight;
polar solvents a1), for which the ratio of water to the total weight of the polar solvent a1) is more than about 80% by weight, preferably more than about 85% by weight and particularly more than about 90% by weight;
polar solvents a1), for which the polar solvent a1) comprises water and ethanol and the ratio by weight of water to ethanol is from about 50:1 to about 1:1, preferably from about 40:1 to about 2:1 and particularly from about 25:1 to about 5:1.

A second significant component of the inventive cosmetic compositions is the care component a2). In relation to the ease of production, applicability and cosmetic effect of the inventive cosmetic compositions, it has proven to be advantageous if the proportion by weight of care component a2) to the total weight of the cosmetic preparation is from about 0.2 to about 18% by weight, preferably from about 0.5 to about 12% by weight and particularly from about 1.0 to about 10% by weight.

Particularly preferred care components are selected from the group comprising
i) cationic polymers;
ii) cationic surfactants;
iii) silicones, especially from the group of
   alkoxylated dimethicones, preferably from the group of ethoxylated dimethicones and especially from the group of compounds with the INCI name of PEG-12 dimethicones;
   Aminofunctional silicones;
iv) polyols;
v) protein hydrolysates, especially from the group of hydrolysates of keratin, silk protein and wheat protein;
vi) waxes.

The cosmetic preparation a) may contain one or more different care components. Cosmetic products, for which the cosmetic preparation comprises at least two, preferably at least three and particularly at least for care components a2), which are different from one another.

Some particularly preferred care combinations are shown in the following Tables:

| | Formula 1 | Formula 2 | Formula 3 | Formula 4 | Formula 5 | Formula 6 |
|---|---|---|---|---|---|---|
| Cationic Polymer | x | x | x | x | x | |
| Cationic Surfactant | x | | | | | x |
| Silicone | | x | | | | x |
| Polyol | | | x | | | |
| Protein Hydrolysate | | | | x | | |
| Wax | | | | | x | |

| | Formula 7 | Formula 8 | Formula 9 | Formula 10 | Formula 11 | Formula 12 |
|---|---|---|---|---|---|---|
| Cationic Polymer | | | | | | |
| Cationic Surfactant | x | x | x | | | |
| Silicone | | | | x | x | x |
| Polyol | x | | | x | | |
| Protein hydrolysate | | x | | | x | |
| Wax | | | x | | | x |

| | Formula 13 | Formula 14 | Formula 15 | Formula 16 | Formula 17 | Formula 18 |
|---|---|---|---|---|---|---|
| Cationic Polymer | | | | x | x | x |
| Cationic Surfactant | | | | x | x | X |
| Silicone | | | | x | | |
| Polyol | x | x | | | x | |
| Protein hydrolysate | x | | x | | | x |
| Wax | | x | x | | | |
| | Formula 19 | Formula 20 | Formula 21 | Formula 22 | Formula 23 | Formula 24 |
| Cationic Polymer | x | x | x | x | x | x |
| Cationic Surfactant | x | | | | | |
| Silicone | | x | x | x | | |
| Polyol | | x | | | x | x |
| Protein Hydrolysate | | | x | | x | |
| Wax | x | | | x | | x |
| | Formula 25 | Formula 26 | Formula 27 | Formula 28 | Formula 29 | Formula 30 |
| Cationic Polymer | x | | | | | |
| Cationic Surfactant | | x | x | x | x | x |
| Silicone | | x | x | x | | |
| Polyol | | x | | | x | x |
| Protein hydrolysate | x | | x | | x | |
| Wax | x | | | x | | x |
| | Formula 31 | Formula 32 | Formula 33 | Formula 34 | Formula 35 | Formula 36 |
| Cationic Polymer | | | | | | x |
| Cationic Surfactant | x | | | | | x |
| Silicone | | x | x | x | | x |
| Polyol | | x | x | | x | x |
| Protein hydrolysate | x | x | | x | x | x |
| Wax | x | | x | x | x | x |

The cationic polymers form a first group of particularly preferred care components a2).

The group of cationic polymers a3) comprises, in particular, the cationic polymers with the INCI names of polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, poly-quaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-50. polyquaternium-55, polyquaternium-56, polyquaternium-67, polyquaternium-68 and polyquaternium-69.

Especially the cationic celluloses are preferred as cationic polymers. Preferred cosmetic products are exemplified in that the care component a2) is selected from the group of cationic cellulose derivatives. Examples of cationic celluloses are the polymers with the trade names of Celquat H 100 and Celquat L 200 (polyquaternium-4), polymer JR 400 (INCI name: polyquaternium-10), polymer LM-200 (INCI name: polyquaternium-24, as well as mirustyle CP (polyquaternium-72). Suitable cationic guar derivatives are sold, for example, under the trade names of jaguar, N-hance, cosmedia guar and aquacat (INCI name: guar hydroxypropyltrimonium chloride).

Components of cationic polymers, which are particularly suitable as contemplated herein, are quaternized hydroxyethylcellulose polymers, which were substituted with trimethylammonium and dimethyldodecylammonium groups. The cationic substitution of the polymers increases the substantivity of the polymers to hair, because the surface of hair is charged negatively, while the hydrophobic modification (the dodecylammonium supports the deposition of further conditioning materials from the hair conditioner on the scalp and on the hair.

Cationic polymers, which are particularly preferred as contemplated herein, have a cationic degree of substitution of from about 0.152 to about 0.25 and a hydrophobic degree of substitution (HS) of about HS 0.01. On the average, the nitrogen content of these cationic polymers in the molecule ranges from about 0.5% to about 3% and preferably from about 0.6% to about 2.7% and, in particular, from about 0.8% to about 2.4% Such polymers are known under the INCI name of polyquaternium 67 and are offered on the market, for example, by the Amerchol company under the names of polymer® SL or polymer® SK. Preferred cosmetic preparations a) contain from about 0.01 to about 10% by weight, preferably from about 0.05 to about 5% by weight and particularly from about 0.1 to about 3.0% by weight of cationic polymer.

The cationic surfactants, especially the cationic surfactants from the group of quaternary ammonium compounds, the esterquats and the amidoamines form a second group of preferred care compounds a2).

Preferred quaternary ammonium compounds are the ammonium halides, especially the chlorides and bromides, such as alkyltrimethylammonium chlorides, dialkyldimethylammonium chlorides and trialkylmethylammonium chlorides, such as cetyltrimethylammonium chloride, stearyl-trimethylammoniumchloride, distearyldimethylammonium chloride, behenyltrimethylammonium chloride, lauryldimethylammonium chloride, lauryldimethylbenzylammonium chloride and tricetylmethylammonium chloride, as well as the imidazolium compounds, which are known under the names of quaternium-27 and quaternium-83. The long chain alkyl groups of the surfactants named above preferably have 10 to 18 carbon atoms Particularly preferred inventive cosmetic preparations a) are exemplified in that they contain (Cu to C18) alkyltrimethylammonium salt(s).Cetyltrimethylammonium and behenyltrimethylammonium salts are particularly preferred. Cetyltrimethylammonium salts and behenyltrimethylammonium salts, in the form of methosulfates and/or chlorides are especially preferred.

The following come into consideration as amidoamines, which may be quaternized and come into consideration as contemplated herein: Tego Amide® S18 (Evonik; INCI name: stearamidopropyl dimethylamines), Lexamine® S 13 (Inolex; INCI name: Stearamidopropyl dimethylamine), Incromine® SB (Croda; INCI name: stearamidopropyl dimethylamines), Witcamine 100 (Witco, INCI name: cocamidopropyl dimethylamines), Incromine BB (Croda, INCI name: behenamidopropyl dimethylamines), Mackine 401 (McIntyre, INCI name: isostearylamidopropyl dimethylamines) and other Mackine types, Adogen 518V (Witco, INCI name: stearylamidopropyl dimethylamines), and as permanent cationic aminoamines: Rewoquat RTM 50 (Witco Surfactants GmbH, INCI name: ricinoleamidopropyltrimonium methosulfate), Empigen CSC (Albright & Wilson, INCI name: cocamidopropyltrimonium chloride), Swanol Lanoquat DES-50 (Nikko, INCI name: quatemium-33), Rewoquat UTM 50 (Witco Surfactants GmbH, undecyleneamidopropyltrimonium methosulfates). Stearamidopropyl dimethylamine is particularly preferred. Stearamidopropyl dimethylamine is particularly preferred.

Preferred cosmetic preparations a) contain from about 0.01 to about 10% by weight, preferably from about 0.05 to about 5% by weight and particularly from about 0.1 to about 3.0% by weight of cationic surfactant.

The silicones, especially the
alkoxylated dimethicones, preferably from the group of ethoxylated/propoxylated dimethicones
aminofunctional silicones
form a third group of preferred care compounds a2).

The group of alkoxylated dimethicones includes, for example,
ethoxylated dimethicones with the INCI name of PEG-x dimethicones with x=2 to 20, preferably from 3 to 17 and particularly 11 or 12;
the ethoxylated dimethicones with the INC I name of bis-PEG-y dimethicones with x=from 3 to 25 and preferably fromm 4 to 20;
the ethoxylated/propoxylated dimethicones with the INCI name of PEG/PPG a/b dimethicones, wherein a and b, independently of one another, represent the members from 2 to 30. preferably from 12 to 24 and particularly from 14 to 20;
the ethoxylated/propoxylated dimethicones with the INCI name of PEG/PPG c/d dimethicones, wherein c and d, independently of one another, represent the numbers from 10 to 25, preferably from 14 to 20 and particularly from 14 to 16;
the ethoxylated/propoxylated dimethicones with the INCI name of bis-PEG/PPG-e/fPEG/PPG g/h dimethicone, wherein e, f, g and h, independently of one another, represent numbers from 10 to 20. preferably from 14 to 18 and particularly 16.

The group of amino-functional silicones, which have at least one amino group that optionally is substituted, are referred to as amodimethicones according to the INCI declaration.

Preferred cosmetic preparations contain one or more amino-functional silicones of the formula M(RₐQ_{b}Si0(_{4-a-b)/2)x}(RbSi0_{(4-02)y}M (Si-1). In this formula
R is a hydrocarbon or a hydrocarbon group with 1 to about 6 carbon atoms, preferably methyl,
Q a polar group of the general formula —R¹HZ, preferably a group of the Formula —CH₂CH₂CH₂ NHCH₂ CH₂ NH₂,
wherein
R¹ a divalent connecting group, which is linked to hydrogen and the Z group and composed of carbon and hydrogen atoms, carbon, hydrogen and oxygen atoms or carbon hydrogen and nitrogen atoms, and
Z is an organic, amino functional group, which contains at least one amino functional group, which preferably is a —NHCH₂CH₂NH₂ NHCH₂CH₂NH₂ or a group of formula CH₂CH₂CH₂—NHCH₂CH₂NH₂
a has values ranging from about 0 to about 2
b has values ranging from about 1 to about 3,
a+b is less than or equal to 3 and
c is a number ranging from about 1 to about 3, and
x is a number ranging from 1 to about 2000. preferably from about 3 to about 50 and particularly from about 3 to about 25, and
y is a number ranging from 20 to about 10.000. preferably from about 125 to about 10.000 and particularly for about 150 to about 1000; and
M is a suitable silicone end group, known in the prior art and preferably trimethylsiloxy.

Examples of R¹ include methylene, ethylene, propylene, hexamethylene, decamethylene, CH₂CH(CH₃)CH₂—, phenylene, naphthylene, —CH₂CH₂SCH₂CH₂—, —CH₂CH₂OCH₂—, —OCH₂CH₂—, —OCH₂ CH₂CH₂—, —CH₂CH(CH₃)C(0)OCH₂—, (CH₂)₃CC(0)OCH₂CH₂—, —C₆H₄C₆H₄—, —C₆H₄CH₂C₆H₄— and (CH₂)₃C(0)SCH₂CH₂—.

Preferred inventive cosmetic preparations contain an amino functional silicone of formula R′aG₃₋ₐ—Si(OSiG₂)n-(0SiG bR′₂-b)m-O-SIG₃₋ₐ-R′ₐ(Si-2), wherein
G is —H, a phenyl group, —OH, -0-CH₃, —CH₃, -0-CH₂CH₃, —CH₂CH₃, -0-CH₂CH₂CH₃, —CH₂CH₂CH₃, -0-CH(CH₃)₂, —CH(CH₃)₂, -0-CH₂CH₂CH₂CH₃, —CH₂CH₂CH₂CH₃,0-CH₂CH(CH₃)₂, —CH₂CH(CH₃)₂, -0-CH(CH₃)CH₂CH₃, —CH(CH₃)CH₂CH₃, -0-C(CH₃)₃, —C(CH₃)₃;
a is a number between 0 and 3, especially 0;
b is a number between 0 and 1, especially 1;
m and n are numbers, the sum of which (m+n) is between 1 and 2000. preferably between 50 and 150, and n preferably having values from 0 to1999 and especially from 49 to 149 and m preferably having values from 1 to 2000 and especially from 1 to 10,
R′ is a monovalent group selected from
   -Q-N(R″)—CH₂—CH₂—N(R″)₂
   -Q-N(R″)₂
   -Q-N1R″)₃A⁻
   -Q-N+H(R″)₂ A⁻
   -Q-N+H₂(R″)A⁻
   -Q-N(R″)—CH₂—CH₂—N+R″H₂A⁻,
wherein each Q represents a chemical bond, —CH₂—, —CH₂—CH₂—, —CH₂CH₂CH₂—, —C(CH₃)₂—, —CH₂CH₂CH₂CH₂—, —CH₂ C(CH₃)₂—, —CH(CH₃)CH₂CH₂—,
R″ representing identical or different groups from the group comprising —H, -phenyl, -benzyl, —CH₂—CH(CH₃)Ph, the C₁₋₂₀ alkyl groups, preferably —CH₃, —CH₂CH₃, —CH₂CH₂CH₃, —CH₃, —CH₂CH₃, —CH₂CH₂CH₃, and A the presenting an anion, which preferably is selected from chloride, bromide, iodide or methosulfate.

Particularly preferred cosmetic preparations are exemplified owing to the fact that they contain at least one amino functional silicone of formula (Si2-a)

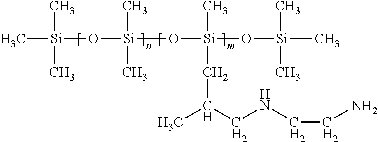

wherein m and n are numbers, the sum of which (m+n) is between 1 and 2000. Preferably between 50 and 150; and n preferably having values from 0 to1999 and especially from 49 to 149 and m preferably having values from 1 to 2000 and especially from 1 to 10.

In a further preferred embodiment, cosmetic preparations contain an amino functional silicone of formula (Si-2b)

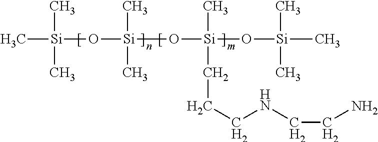

in which m and n are numbers, the sum of which (m+n) is between 1 and 2000. preferably between 50 and 150. and n preferably has values from 0 to1999 and especially from 49 to 149 and m preferably has values from 1 to 2000 and especially from 1 to 10.

These silicones of formula (Si2-a) and (Si2-b) are referred to as amodimethicone according to the INCI declaration. Corresponding amodimethnicones are obtainable, for example, in the form of an emulsion as a commercial product, Dow Corning 939 or as a commercial product, Corning 949.

In an alternative embodiment, the amino functional silicone a2) however is a functionalized amodimethicone. Corresponding functionalized amodimethicones are described, for example, by the formula (Si-2c)

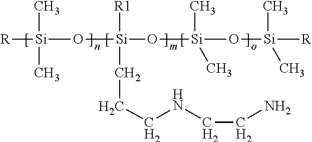

wherein
R represents —OH an (optionally ethoxylated and/or propoxylated) (C₁ to C₂₀) alkoxy group or a CH₃ group,
R1 represents —OH, a (C₁ to C₂₀) alkoxy group or a —CH₃ group and
m, n1 and n2 are numbers, the sum of which (m+n1+n2) is between 1 and 2000. Preferably between 50 and 150 and (n1+n2) preferably assumes values from 0 to1999 and especially from 49 to 149 and m preferably has values from 1 to 2000 and especially from 1 to 10.

Amino functional silicone a2) from the group of hydroxylamino-modified silicones, for which, in the above formula (Si-2b) R represents OH, are preferred. Corresponding silicones with the INCI name of amodimethicones are obtainable as a commercial product, Belsil ADM 652, from Wacker.

If the R group represents (an optionally ethoxylated and/or propoxylated) (C1 to C20) alkoxy group, then, for example, bis(C13 to C15 alkoxy) PG amodimethicone (for example, as a commercial product) is included with this general formula (Si-2b) for example bis(C13-15) alkoxy) PG amodimethicone (obtainable, for example as a commercial product: DC 8500 from Dow Corning), trideceth-9 PG-amodimethicone (for example as a commercial product, Silcare Silicone SEA of the Clariant company) are included with this general formula (Si-2b).

Regardless of the amino functional silicones, which are used, inventive cosmetic preparations are preferred, which contain an amino functional silicone, the amine number of which is greater than 0.25 meq/g, preferably greater than 0.3 meq/g and particularly greater than 0.4 meq/g. The amine number represents the milliequivalents of amine per gram of the amino functional silicone. It can be determined by titration and also stated in the unit of mg KOH/g.

Preferred cosmetic preparations a) contain from about 0.01 to about 30% by weight, preferably from about 0.05 to about 20% by weight and particularly form about 0.1 to about 5.0% by weight of silicone.

The polyols form a fourth group of preferred care materials a2). The polyol is different from the polar solvent a1). Especially the polyols from the group comprising glycerin, 1,2-ethanol, polyethylene glycols with a MW>400. propanediol, butane diol, especially 1,3-butanediol, hexanediol, particularly 1,6-hexanediol, sorbitol, threitol, erythritol, arabitol, altritol, ribitol, xylitol, galactitol, mannitol, iditol and panthenol are suitable as polyols. Glycerin, sorbitol and panthenol are particularly suitable polyols.

The polyols may be used as individual substances or in the form of polyol mixtures. Preferred inventive materials are distinguished by the fact that they contain fewer than four, preferably 1 to 3 polyols.

Preferred cosmetic preparations a) contain from about 0.01 to about 20% by weight, preferably from about 0.05 to about 10% by weight and particularly from about 0.1 to about 5.0% by weight of polyol.

The protein hydrolysates form the fifth group of preferred care materials a2). The group of particularly preferred natural protein hydrolysates, that is, the hydrolysates of natural proteins, can be divided into protein hydrolysates of plant and of animal origin.

Elastin, collagen, keratin, silk and milk protein hydrolysates, which may also be present in the form of salts are, for example, animal protein hydrolysates. Such products are sold, for instance, under the trade names of Dehylan® (Cognis), Promois® (Interorgana), Collapuron® (Cognis), Nutrilan° (Cognis), Gelita-Sol® (Deutsche Gelatine Fabriken Stoess & Co), Lexein° (Inolex) and Kerasol° (Croda).

Soybean, almond, pea, potato and wheat protein hydrolysates are vegetable protein hydrolysates Such products may be obtained, for instance, under the trade names of Gluadin° (Cognis), DiaMin® (Diamalt), Lexein° (Inolex), Hydrosoy® (Croda), Hydrolupin® (Croda), Hydrosesame® (Croda), Hydrotritium® (Croda) and Crotein° (Croda).

The group of marine protein hydrolysates comprises protein hydrolysates of animal and vegetable origin. Marine protein hydrolysates include, for example, collagen hydrolysates from fish or algae, protein hydrolysates from sea shells and pearl hydrolysates. The mixtures of substances, sold under the trade names of Pearl Protein Extract BG(RF) or Crodarom® Pearl are examples of the inventive pearl hydrolysates or pearl extracts.

Particularly preferred are protein hydrolysates based on collagen, silk protein, keratin, milk protein, soy protein, almond protein, wheat protein or pearls. Most preferred is the use of hydrolysates of keratin, soy protein and wheat protein.

Preferred cosmetic preparations a) contain from about 0.01 to about 10% by weight, preferably from about 0.05 to about 5.0% by weight and particularly from about 0.05 to about 5.0% by weight and more particularly from about 0.1 to about 3.0% of protein hydrolysate.

The waxes form the last group of preferred care components a2) The wax a2) may be of natural or synthetic origin. Preferred waxes melt above about 40° C., particularly above about 50° C. and more particularly at temperatures between about 50° C. and about 90° C.

As waxes a2), solid waxes or iso-paraffins, plant waxes such as candelillaWax, carnauba Wax, Esparto grass Wax, Japan wax, cork wax, sugarcane wax, ouricury wax, montan wax, sunflower wax, fruit waxes and animal waxes, such as beeswax and other insect waxes, spermaceti, shellac wax, will wax and barzel that, furthermore mineral waxes, such as ceresin and ozokerit or the petrochemical waxes such as petrolatum, paraffin waxes, Micro waxes of polyethylene or polypropylene and polyethylene glycol waxes may be used. It may be advantageous to use hydrogenated or hardened waxes. Furthermore, chemically modified waxes, especially the hard waxes, such as montan ester waxes sasol waxes and hydrogenated Japan waxes may also be used.

Furthermore suitable are the triglycerides of saturated and optionally hydroxylated C16-30 fatty acids, such as hydrogenated triglyceride fats (hydrogenated palm oil, hydrogenated coconut oil, hydrogenated castor oil), glycerol tribehenate or glycerol tri-12-hydroxystearate, furthermore synthetic full esters of fatty acids and glycols (such as Syncrowax® or polyols with 2-6 carbon atoms, esters of saturated and/or unsaturated branched and/or linear alkane carboxylic acids with a chain length of 1 to 80 carbon atoms and saturated and/or unsaturated, branched and/or linear alcohols with a chain length of 1 to 80 carbon atoms, including, for example, fatty acid, fatty alcohol esters such as stearyl stearate or cetyl palmitate, esters of aromatic carboxylic acids, dicarboxylic acids or hydroxycarboxylic acids (such as 12-hydroxystearic acid) and saturated and/or unsaturated branched and/or linear alcohols having a chain length of 1 to 80 carbon atoms, lactides of long chain hydroxy carboxylic acids and full esters of fatty alcohols and dicarboxylic and tricarboxylic acids, such as dicetyl succinate or dicetyl/stearyl adipate and mixtures of these substances.

The wax components may also be selected from the group of esters of saturated, linear alkane carboxylic acids having a chain length of 14 to 44 carbon atoms and saturated, linear alcohols having a chain length of 14 to 44 carbon atoms, if the wax components or the totality of wax components are solid at room temperature. The wax components may be selected, for example, from the group of C16-C36 alkyl stearates, C10-C40 alkyl stearates, C2-C40 alkyl isostearates, C20-C40 dialkyl esters of dimeric acids, C18-C38 alkyl hydroxystearoyl stearates, C20-C40 alkyl, erucates and, furthermore, C30-C50 alkyl beeswax as well as cestearyl beeswax may be used. Silicone waxes, such as stearyl trimethylsilane/stearyl alcohol may also be advantageous. The esters of saturated, univalent C20-C60 alcohols and saturated C8-C30 monocarboxylic acids, especially a C20-C40 alkyl stearate, which may be obtained under the name of KesterWax® K82H from Koster Keunen Inc. are preferred wax components. The wax or wax components should be solid at about 25° C., yet melt at temperatures ranging from about 35° to about 95° C., a range of about 45° to about 85° C. being preferred.

Further preferred wax components are fatty alcohols. For example, stearyl alcohol, cetyl alcohol, lauryl alcohol, myristyl alcohol, arachidyl alcohol, capryl alcohol, capric alcohol and behenyl alcohol may be used as fatty alcohols.

Preferably, the wax a2) is selected from beeswax (Cera Alba), carnauba wax, candelilla wax, montan wax, cetyl palmitate, microcrystalline waxes (microcrystalline paraffins) and mixtures thereof. Especially preferred is the use of a care component a2) from the group of beeswax (Cera Alba), carnauba wax and microcrystalline waxes (microcrystalline paraffins).

Natural, chemically modified and synthetic waxes may be used alone or in combination. The inventive teachings also include the combined use of several waxes. Furthermore, a series of wax mixtures, optionally mixed with further additives, are also commercially available. The waxes, obtainable under the names of “Special Wax 7686 OE” (a mixture of cetyl palmitate, beeswax, microcrystalline wax and polyethylene with a melting point range from about 73° to about 75° C.; manufactured by Kahl & Co), Polywax® GP 200 (a mixture of stearyl alcohol and polyethylene glycol stearate with a melting point of from about 47° to about 51° C., manufactured by (Croda) and “Weichceresin® FL 400” (a Vaseline/Vaselinol/wax mixture with a melting point of from about 50° to about 54° C.; manufactured by Parafluid Mineralolgesellschaft) are examples of mixtures preferably used as contemplated herein. A further particularly preferred mixture of waxes a2) comprises bees wax and carnauba wax, optionally in combination with microcrystalline wax.

Preferred cosmetic preparations a) contain from about 0.01 to about 10% by weight, preferably from about 0.05 to about 5% by weight and particularly from about 0.1 to about 3.0% by weight of wax.

Aside from the cosmetic preparation a), the inventive cosmetic products comprise furthermore a device for flash evaporation. As contemplated herein, the expression “flash evaporation” refers to the formation of vapor when the pressure is lowered in a closed space, which is filled with a liquid and is under pressure in comparison to the surroundings. An appropriate pressure can be produced, for example, in that an amount of cosmetic preparation a) is heated in a closed space to a temperature T1. In the closed space, the liquid has a saturation pressure p1 at a given temperature T1. If the closed space is opened, for example, by means of a valve to a relaxation space, which is not under pressure, with the pressure p0<p1, the pressure in the previously closed space drops and, as the new pressure level spreads, the cosmetic preparation a) or the solvent or parts of the solvent, contained in the cosmetic preparation, are evaporated. The resulting vapor or spray mist formed can be used for the application of specific cosmetic preparations.

If, starting out from standard conditions (T0=25° C., p0=1,000 bar), the cosmetic preparation a) is heated in a closed space, aside from an elevated temperature, an elevated pressure of the cosmetic preparation a) results. This elevated pressure can be relieved in a relaxation space to a pressure p0, for example, to the pressure of the surrounding area (p0=10.00 bar), as a result of which at least a partial evaporation of the cosmetic preparation a) is attained.

The pressure on the cosmetic preparation a) can be relieved directly in the space, in which the cosmetic preparation a) was heated previously. Alternatively, the cosmetic preparation a), which is heated and under pressure, can also be transported into a second space, in which the pressure is then relieved subsequently.

In other words, flash evaporation is a method, for which the cosmetic preparation a) is heated in a closed container by a heater to temperatures above the surrounding temperature; a pressure above the surrounding pressure results in the container and the pressure on the heated cosmetic preparation a) is subsequently relieved out of the container into the surroundings.

Accordingly, a device for flash evaporation is a device, which comprises a container and a heater and is equipped in such a manner, that a cosmetic preparation a) in the closed container can be heated by means of the heater to temperatures above the surrounding temperature in such a way, that a pressure above the surrounding pressure develops in the container and the pressure on the heated cosmetic preparation a) can be relieved from the container into the surroundings.

Simultaneously with relieving the pressure or thereafter, the cosmetic preparation a) may be supplied to a nozzle, by means of which properties of the vapor or spray mist, produced by the flash evaporation, especially the droplet size or the droplet density, but also the width of the spray and the shape of the spray cone can be affected. The use of nozzles, preferably atomizing nozzles, therefore is preferred. The specific type or configuration of the nozzle can be established selectively, depending on the properties of the respective spray mist.

To summarize, a preferred device for the flash evaporation has
b1) a container b1), which can be opened and closed by means of a valve and which defines a closed inner space, in which the cosmetic preparation can be accommodated,
b2) a heating device b2), which enables a cosmetic preparation in the container b1) to be heated.

The use of an additional nozzle b3), which enables the cosmetic preparation a), escaping from the container, to be atomized is especially preferred. Alternatively to a valve, a closing element, which has a comparable action and closes off or releases an associated opening in the container by an appropriate positional change, can also be used.

A preferred object of the present disclosure accordingly is a cosmetic product, comprising
a) a cosmetic preparation containing, based on its total weight,
   a1) from about 70 to about 99% by weight of a polar solvent, wherein;
   a2) at least about 0.1% by weight of care component;
b) a device for the flash evaporation of the cosmetic preparation a), the device for the flash evaporation comprising a container b1) and a heater b2 and being configured in such a manner that
   the cosmetic preparation a) can be accommodated in the interior of the container b1),
   the interior of the container b1), which is at least partially filled with the cosmetic preparation a), can be closed off,
   the cosmetic preparation a) in the closed interior of the container b1) can be heated with the heater b2) while the pressure increases.

A further preferred object of the present disclosure accordingly is a cosmetic product, comprising
a) a cosmetic preparation containing, based on its total weight,
   a1) from about 70 to about 99% by weight of a polar solvent, wherein;
   a2) at least about 0.1% by weight of care component;
b) a device for the flash evaporation of the cosmetic preparation a)
   b1) a container b1), which can be closed off and opened up by means of a valve,
   b2) a heating device, which enables a cosmetic preparation in the container b1) to be heated, and
   b3) and a nozzle b3), which enables the cosmetic preparation a) to be atomized.

An especially preferred object of the present disclosure accordingly is a cosmetic product, comprising
a) a cosmetic preparation containing, based on its total weight,
   a1) from about 70 to about 99% by weight of a polar solvent, wherein
   a2) at least about 0.1% by weight of care component;
b) a device for the flash evaporation of the cosmetic preparation a), the device for the flash evaporation comprising a container b1) and a heater b2 and being configured in such a manner that
   the cosmetic preparation a) can be accommodated in the interior of the container b1),
   the interior of the container b1), which is at least partially filled with the cosmetic preparation a), can be closed off,
   the cosmetic preparation a) in the closed interior of the container b1) can be heated with the heater b2) while the pressure increases,
   the pressure on the heated cosmetic preparation a) from the interior of the container b1) can be lowered by being relieved into the surroundings.

The container b1), in which the cosmetic preparation is heated, is configured in such a way that it enables this container to be closed off completely from the environment while the cosmetic preparation a) is being heated and, after the heating, to be opened up to enable the cosmetic preparation a) to be flash evaporated. This may be ensured, for example, by a component for controlling flow, especially by a valve.

Preferably, the container b1), in which the cosmetic preparation is heated, is in contact with a further container, from which the amount of cosmetic preparation, which is to be flash evaporated, is transferred before being heated in container b1). The access between this reservoir and the container b1) can therefore be opened and closed by an appropriate device, such as a valve. This further container preferably is in the form of a reservoir, that is, it preferably comprises a multiple, such as more than about 10× and preferably more than about 50× of the amount of cosmetic preparation necessary for one evaporation process. In other words, the capacity of the further container/reservoir preferably is a multiple, for example, more than about 10 times, preferably more than about 20 times and particularly more than about 50 times the capacity of the container b1).

A further preferred object of the present disclosure accordingly is a cosmetic product, comprising
a) a cosmetic preparation containing, based on its total weight,
   a1) from about 70 to about 99% by weight of a polar solvent, wherein
   a2) at least about 0.1% by weight of care component;
b) a device for the flash evaporation of the cosmetic preparation a), comprising
   b1) a container b1), which can be closed off and opened by means of a valve,
   b2) a heating device, which enables a cosmetic preparation in the closed container b1) to be heated,
   b3) and a nozzle b3), which enables the cosmetic preparation a) to be atomized;
c) and a reservoir for the cosmetic preparation a), from which the cosmetic preparation a) can reach the container b1),
   wherein the access between the reservoir and the container b1) has a flow-regulating component, by means of which the flow of the cosmetic preparation a) from the reservoir into the container b1) can be interrupted;
   the capacity of the reservoir is at least about 10 fold, preferably at least about 20 fold and particularly at least about 50 fold that of the container b1).

The reservoir is not a pressure container and the cosmetic preparation in the reservoir is not under pressure; in other words, the pressure in the interior of the reservoir corresponds to the atmospheric pressure (air pressure or atmospheric pressure). For example, appropriate cosmetic products do not include, for example, any propellant. The cosmetic product also does not have a pump device, which is suitable for releasing or spraying the cosmetic preparation into the environment without the action of a device for flash evaporation.

An especially preferred object of the present disclosure accordingly is a cosmetic product, comprising
a) a cosmetic preparation containing, based on its total weight,
   a1) from about 70 to about 99% by weight of a polar solvent, wherein
   a2) at least about 0.1% by weight of care component;
b) a device for the flash evaporation of the cosmetic preparation a), comprising
   b1) a container b1), which can be closed off and opened up by means of a valve
   b2) a heating device, which enables a cosmetic preparation in the closed container b1) to be heated
   b3) and a nozzle b3), which enables the cosmetic preparation a) to be atomized;
c) and a reservoir for the cosmetic preparation a), from which the cosmetic preparation a) can reach the container b1),
   wherein the access between the reservoir and the container b1) has a flow-regulating component, by means of which the flow of the cosmetic preparation a) from the reservoir into the container b1) can be interrupted;
   the capacity of the reservoir is at least about 10 fold and preferably at least about 50 fold that of the container b1);
   the pressure in the interior of the reservoir corresponds to that of the surroundings.

An especially preferred object of the present disclosure accordingly is a cosmetic product, comprising
a) a cosmetic preparation containing, based on its total weight,
   a1) from about 70 to about 99% by weight of a polar solvent, wherein;
   a2) at least about 0.1% by weight of care component;
b) a device for the flash evaporation of the cosmetic preparation a), comprising
   b1) a container b1), which can be closed off and opened by means of a valve
   b2) a heating device, which enables a cosmetic preparation in the closed container b1) to be heated
   b3) and a nozzle b3), which enables the cosmetic preparation a) to be atomized;
c) and a reservoir for the cosmetic preparation a), from which the cosmetic preparation a) can reach the container b1),
   wherein the access between the reservoir and the container b1) has a flow-regulating component, by means of which the flow of the cosmetic preparation a) from the reservoir into the container b1) can be interrupted;
   the capacity of the reservoir is at least about 10 fold and preferably at least about 50 fold that of the container b1);
   the pressure in the interior of the reservoir corresponds to the surrounding pressure and the cosmetic product does not include a propellant.

Furthermore, cosmetic products are preferred, which comprise
a) a cosmetic preparation containing, based on its total weight,
   a1) from about 70 to about 99% by weight of a polar solvent, wherein
   a2) at least about 0.1% by weight of care component;
b) a device for the flash evaporation of the cosmetic preparation a), comprising
   b1) a container b1), which can be closed off and opened by means of a valve
   b2) a heating device, which enables a cosmetic preparation in the closed container b1) to be heated
   b3) and a nozzle b3), which enables the cosmetic preparation a) to be atomized;
c) and a reservoir for the cosmetic preparation a), from which the cosmetic preparation a) can reach the container b1),
   wherein the access between the reservoir and the container b1) has a flow-regulating component, by means of which the flow of the cosmetic preparation a) from the reservoir into the container b1) can be interrupted;
   the capacity of the reservoir is at least 10 fold and preferably at least 50 fold that of the container b1);
   the pressure in the interior of the reservoir corresponds to atmospheric pressure,
the cosmetic product not having a pump device, which is suitable for releasing or spraying the cosmetic preparation a) without the action of a device for flash evaporation.

To summarize, a particularly preferred object of the present disclosure accordingly is a cosmetic product, comprising
a) a cosmetic preparation containing, based on its total weight,
   a1) from about 70 to about 99% by weight of a polar solvent, and
   a2) at least about 0.1% by weight of care component;
b) a device for the flash evaporation of the cosmetic preparation a)
   b1) a container b1), which can be closed off and opened up by means of a valve
   b2) a heating device, which enables a cosmetic preparation in the closed container b1) to be heated
   b3) and a nozzle b3), which enables the cosmetic preparation a) to be atomized;
c) and a reservoir for the cosmetic preparation a), from which the cosmetic preparation a) can reach the container b1),
   wherein the access between the reservoir and the container b1) has a flow-regulating component, by means of which the flow of the cosmetic preparation a) from the reservoir into the container b1) can be interrupted;
   the capacity of the reservoir is at least 10 fold and preferably at least about 50 fold that of the container b1);
   the pressure in the interior of the reservoir corresponds to the atmospheric pressure and the cosmetic product does not include a propellant;
the cosmetic product does not have a pump device, which is suitable for releasing or spraying the cosmetic preparation a) without the action of a device for flash evaporation.

Aside from the above-described components a1) and a2), the inventive cosmetic materials may contain further active ingredients, additives and care materials.

In a preferred embodiment, the cosmetic preparation a) furthermore contains a non-ionic surfactant a5), preferably a non-ionic surfactant from the group comprising PEG derivatives of hydrogenated castor oil, PEG-40 hydrogenated castor oil being particularly preferred. Particularly preferred non-ionic surfactants are PEG derivatives of hydrogenated castor oil, which may be obtained, for example, under the name of PEG hydrogenated castor oil such as PEG-30 hydrogenated castor oil, PEG-33 hydrogenated castor oil, PEG-35 hydrogenated castor oil, PEG 36 hydrogenated castor oil or PEG-40 hydrogenated castor oil. As contemplated herein, the use of PEG-40 hydrogenated castor oil is particularly preferred. The proportion by weight of the non-ionic surfactant, especially of PEG-40 hydrogenated castor oil to the total weight of the cosmetic preparation a) preferably is sealed from about 0.05 to about 2.0% by weight, especially from about 0.1 to about 1.5% by weight and, particularly from about 0.2 to about 1.0% by weight.

The composition of some particularly preferred, inventive, cosmetic preparations is given in the following Tables (unless stated otherwise, the data in weight percent is based on the total weight of the cosmetic material). Reference is made to further preferred embodiments of these particularly preferred compositions, mutatis mutandis, to the inventive cosmetic preparations a) previously described for the cosmetic preparations. It is to be appreciated that all values in the referenced ranges are approximations and should be read as “about” the stated value in the table.

| | Formula 1 | Formula 2 | Formula 3 | Formula 4 | Formula 5 |
|---|---|---|---|---|---|
| Polar solvent a1) | 80 to 99 | 90 to 98 | 92 to 97 | 95.8 | 94.4 |
| Cationic polymer | 0.10 to 10 | 0.05 to 50. | 0.1 to 30. | 0.3 | 0.8 |
| Cationic surfactant | 0.10 to 10 | 0.05 to 50. | 0. to 30. | 0.5 | 0.2 |
| Optional additives | to 100 | to 100 | to 100 | to 100 | to 100 |

| | Formula 6 | Formula 7 | Formula 8 | Formula 9 | Formula 10 |
|---|---|---|---|---|---|
| Polar solvent a1) | 80 to 99 | 90 to 98 | 92 to 97 | 95.8 | 94.4 |
| Cationic polymer | 0.10 to 10 | 0.05 to 5.0 | 0.1 to 3.0 | 0.3 | 0.8 |
| Silicone | 0.10 to 30 | 0.05 to 20 | 0.1 to 10 | 0.3 | 3.3 |
| Optional additives | to 100 | to 100 | to 100 | to 100 | to 100 |

| | Formula 11 | Formula 12 | Formula 13 | Formula 14 | Formula 15 |
|---|---|---|---|---|---|
| Polar solvent a1) | 80 to 99 | 90 to 98 | 92 to 97 | 95.8 | 94.4 |
| Cationic polymer | 0.10 to 10 | 0.05 to 50. | 0.1 to 30. | 0.3 | 0.8 |
| Polyol | 0.10 to 20 | 0.05 to 10 | 0.1 to 50. | 0.2 | 0.1 |
| Optional additives s | to 100 | to 100 | to 100 | to 100 | to 100 |
| | Formula 16 | Formula 17 | Formula 18 | Formula 19 | Formula 20 |
| Polar solvent a1) | 80 to 99 | 90 to 98 | 92 to 97 | 95.8 | 94.4 |
| Protein hydrolysate | 0.10 to 10 | 0.05 to 5.0 | 0.1 to 30. | 0.3 | 0.8 |
| Protein Hydrolysate | 0.10 to 10 | 0.05 to 5.0 | 0. to 3.0 | 0.1 | 0.2 |
| Optional additives | to 100 | to 100 | to 100 | to 100 | to 100 |

| | Formula 21 | Formula 22 | Formula 23 | Formula 24 | Formula 25 |
|---|---|---|---|---|---|
| Polar Solvent a1) | 80 to 99 | 90 to 98 | 92 to 97 | 95.8 | 94.4 |
| Cationic polymer | 0.10 to 10 | 0.05 to 5.0 | 0.1 to 3.0 | 0.3 | 0.8 |
| Wax | 0.10 to 10 | 0.05 to 50. | 0.1 to 3.0 | 1.0 | 1.3 |
| Optional additives | to 100 | to 100 | to 100 | to 100 | to 100 |

| | Formula 26 | Formula 27 | Formula 28 | Formula 29 | Formula 30 |
|---|---|---|---|---|---|
| Polar Solvent a1) | 80 to 99 | 90 to 98 | 92 to 97 | 95.8 | 94.4 |
| Cationic Surfactant | 0.10 to 10 | 0.05 to 5.0 | 0.1 to 3.0 | 0.5 | 0.2 |
| Silicone | 0.10 to 30 | 0.05 to 20 | 0. to 10 | 0.3 | 3.3 |
| Optional additives | to 100 | to 100 | to 100 | to 100 | to 100 |
| | Formula 31 | Formula 32 | Formula 33 | Formula 34 | Formula 3 5 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Polyol | 0.10 to 20 | 0.05 to 10 | 0.1 to 50. | 0.2 | 0.1 |
| Optional additives | to 100 | to 100 | to 100 | to 100 | to 100 |
| | Formula 36 | Formula 37 | Formula 38 | Formula 39 | Formula 40 |
| Polar solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |
| | Formula 41 | Formula 42 | Formula 43 | Formula 44 | Formula 45 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Wax | 0.10 to 10 | 0.05 to 50. | 0.1 to 30. | 10. | 1.3 |
| Optional additives | to 100 | to 100 | to 100 | to 100 | to 100 |

| | Formula 46 | Formula 47 | Formula 48 | Formula 49 | Formula 50 |
|---|---|---|---|---|---|
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 3.3 |
| Polyol | 0.10 to 20 | 0.05 to 10 | 0.1 to 50. | 0.2 | 0.1 |
| Optional additives | to 100 | to 100 | to 100 | to 100 | to 100 |

| | Formula 51 | Formula 52 | Formula 53 | Formula 54 | Formula 55 |
|---|---|---|---|---|---|
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 3.3 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |

| | Formula 56 | Formula 57 | Formula 58 | Formula 59 | Formula 60 |
|---|---|---|---|---|---|
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 3.3 |
| Wax | 0.10 to 10 | 0.05 to 50. | 0.1 to 30. | 10. | 1.3 |
| Optional additives | to 100 | to 100 | to 100 | to 100 | to 100 |

| | Formula 61 | Formula 62 | Formula 63 | Formula 64 | Formula 65 |
|---|---|---|---|---|---|
| Polar Solvent a1) | 80 to 99 | 90 to 98 | 92 to 97 | 95.8 | 94.4 |
| Protein Hydrolysate | 0.10 to 10 | 0.05 to 50. | 0.1 to 30. | 0.1 | 0.2 |
| Wax | 0.10 to 10 | 0.05 to 50. | 0.1 to 30. | 10. | 1.3 |
| Optional additives | to 100 | to 100 | to 100 | to 100 | to 100 |
| | Formula 66 | Formula 67 | Formula 68 | Formula 69 | Formula 70 |
| Polar Solvent a1) | 80 to 99 | 90 to 98 | 92 to 97 | 95.8 | 94.4 |
| Cationic Polymer | 0.10 to 10 | 0.05 to 50. | 0.1 to 30. | 0.3 | 0.8 |
| Cationic Surfactant | 0.10 to 10 | 0.05 to 50. | 0.1 to 30. | 0.5 | 0.2 |
| Silicone | 0.10 to 30 | 0.05 to 20 | 0.1 to 10 | 0.3 | 3.3 |
| Optional additives | to 100 | to 100 | to 100 | to 100 | to 100 |
| | Formula 71 | Formula 72 | Formula 73 | Formula 74 | Formula 75 |
| Polar Solvent a1) | 80 to 99 | 90 to 98 | 92 to 97 | 95.8 | 94.4 |
| Cationic Polymer | 0.10 to 10 | 0.05 to 50. | 0.1 to 30. | 0.3 | 0.8 |
| Cationic Surfactant | 0.10 to 10 | 0.05 to 50. | 0.1 to 30. | 0.5 | 0.2 |
| Polyol | 0.10 to 20 | 0.05 to 10 | 0.1 to 50. | 0.2 | 0.1 |
| Optional additives | to 100 | to 100 | to 100 | to 100 | to 100 |
| | Formula 76 | Formula 77 | Formula 78 | Formula 79 | Formula 80 |
| Polar Solvent a1) | 80 to 99 | 90 to 98 | 92 to 97 | 95.8 | 94.4 |
| Cationic Polymer | 0.10 to 10 | 0.05 to 50. | 0.1 to 30. | 0.3 | 0.8 |
| Cationic Surfactant | 0.10 to 10 | 0.05 to 50. | 0.1 to 30. | 0.5 | 0.2 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |

| | | | |
|---|---|---|---|
| Formula 81 | Formula 82 | Formula 83 | Formula 84 Formula 85 |

| Polar Solvent a1) | 80 to 99 | 90 to 98 | 92 to 97 | 95.8 | 94.4 |
|---|---|---|---|---|---|
| Cationic Polymer | 0.10 to 10 | 0.05 to 50. | 0.1 to 30. | 0.3 | 0.8 |
| Cationic Surfactant | 0.10 to 10 | 0.05 to 50. | 0.1 to 30. | 0.5 | 0.2 |
| Wax | 0.10 to 10 | 0.05 to 50. | 0.1 to 30. | 10. | 1.3 |
| Optional additives | to 100 | to 100 | to 100 | to 100 | to 100 |
| | Formula 86 | Formula 87 | Formula 88 | Formula 89 | Formula 90 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Silicone | 0.10 to 30 | 0.05 to 20 | 0.1 to 10 | 0.3 | 3.3 |
| Polyol | 0.10 to 20 | 0.05 to 10 | 0.1 to 50. | 0.2 | 0.1 |
| Optional additives | to 100 | to 100 | to 100 | to 100 | to 100 |
| | Formula 91 | Formula 92 | Formula 93 | Formula 94 | Formula 95 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Silicone | 0.10 to 30 | 0.05 to 20 | 0.1 to 10 | 0.3 | 3.3 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |
| | Formula 96 | Formula 97 | Formula 98 | Formula 99 | Formula 100 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Silicone | 0.10 to 30 | 0.05 to 20 | 0.1 to 10 | 0.3 | 3.3 |
| Wax | 0.10 to 10 | 0.05 to 50. | 0.1 to 30. | 10. | 1.3 |
| Optional additives | to 100 | to 100 | to 100 | to 100 | to 100 |
| | Formula 101 | Formula 102 | Formula 103 | Formula 104 | Formula 105 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Polyol | 0.10 to 20 | 0.05 to 10 | 0.1 to 50. | 0.2 | 0.1 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |
| | Formula 106 | Formula 107 | Formula 108 | Formula 109 | Formula 110 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Polyol | 0.10 to 20 | 0.05 to 10 | 0.1 to 50. | 0.2 | 0.1 |
| Wax | 0.10 to 10 | 0.05 to 50. | 0.1 to 30. | 10. | 1.3 |
| Optional additives | to 100 | to 100 | to 100 | to 100 | to 100 |
| | Formula 111 | Formula 112 | Formula 113 | Formula 114 | Formula 115 |
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Protein Hydrolysate | 0.10 to 10 | 0.05 to 50. | 0.1 to 30. | 0.1 | 0.2 |
| Wax | 0.10 to 10 | 0.05 to 50. | 0.1 to 30. | 10. | 1.3 |
| Optional additives | to 100 | to 100 | to 100 | to 100 | to 100 |
| | Formula 116 | Formula 117 | Formula 118 | Formula 119 | Formula 120 |
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 3.3 |
| Polyol | 0.10 to 20 | 0.05 to 10 | 0.1 to 50. | 0.2 | 0.1 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |

| | Formula 121 | Formula 122 | Formula 123 | Formula 124 | Formula 125 |
|---|---|---|---|---|---|
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 3.3 |
| Polyol | 0.10 to 20 | 0.05 to 10 | 0.1 to 50. | 0.2 | 0.1 |
| Wax | 0.10 to 10 | 0.05 to 50. | 0.1 to 30. | 10. | 1.3 |
| Optional additives | to 100 | to 100 | to 100 | to 100 | to 100 |

| | Formula 126 | Formula 127 | Formula 128 | Formula 129 | Formula 130 |
|---|---|---|---|---|---|
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 3.3 |
| Protein Hydrolysate | 0.10 to 10 | 0.05 to 50. | 0.1 to 30. | 0.1 | 0.2 |
| Wax | 0.10 to 10 | 0.05 to 50. | 0.1 to 30. | 10. | 1.3 |
| Optional additives | to 100 | to 100 | to 100 | to 100 | to 100 |
| | Formula 131 | Formula 132 | Formula 133 | Formula 134 | Formula 135 |
| Polar Solvent a1) | 80 to 99 | 90 to 98 | 92 to 97 | 95.8 | 94.4 |
| Polyol | 0.10 to 20 | 0.05 to 10 | 0.1 to 50. | 0.2 | 0.1 |
| Protein Hydrolysate | 0.10 to 10 | 0.05 to 50. | 0.1 to 30. | 0.1 | 0.2 |
| Wax | 0.10 to 10 | 0.05 to 50. | 0.1 to 30. | 10. | 1.3 |
| Optional additives | to 100 | to 100 | to 100 | to 100 | to 100 |
| | Formula 136 | Formula 137 | Formula 138 | Formula 139 | Formula 140 |
| Polar Solvent a1) | 80 to 99 | 90 to 98 | 92 to 97 | 95.8 | 94.4 |
| Cationic Polymer | 0.10 to 10 | 0.05 to 50. | 0.1 to 30. | 0.3 | 0.8 |
| Cationic Surfactant | 0.10 to 10 | 0.05 to 50. | 0.1 to 30. | 0.5 | 0.2 |
| Silicone | 0.10 to 30 | 0.05 to 20 | 0.1 to 10 | 0.3 | 2.3 |
| Polyol | 0.10 to 20 | 0.05 to 10 | 0.1 to 50. | 0.2 | 0.1 |
| Protein Hydrolysate | 0.10 to 10 | 0.05 to 50. | 0.1 to 30. | 0.1 | 0.2 |
| Wax | 0.10 to 10 | 0.05 to 50. | 0.1 to 30. | 10. | 1.3 |
| Optional additives | to 100 | to 100 | to 100 | to 100 | to 100 |

| | Formula 141 | Formula 142 | Formula 143 | Formula 144 | Formula 145 |
|---|---|---|---|---|---|
| Polar Solvent a1) | 80 to 99 | 90 to 98 | 92 to 97 | 95.8 | 94.4 |
| Cationic Polymer | 0.10 to 10 | 0.05 to 50. | 0.1 to 30. | 0.3 | 0.8 |
| Optional additives | to 100 | to 100 | to 100 | to 100 | to 100 |

| | Formula 146 | Formula 147 | Formula 148 | Formula 149 | Formula 150 |
|---|---|---|---|---|---|
| Polar Solvent a1) | 80 to 99 | 90 to 98 | 92 to 97 | 95.8 | 94.4 |
| Cationic Surfactant | 0.10 to 10 | 0.05 to 50. | 0.1 to 30. | 0.5 | 0.2 |
| Optional additives | to 100 | to 100 | to 100 | to 100 | to 100 |

| | Formula 151 | Formula 152 | Formula 153 | Formula 154 | Formula 155 |
|---|---|---|---|---|---|
| Polar Solvent a1) | 80 to 99 | 90 to 98 | 92 to 97 | 95.8 | 94.4 |
| Silicone | 0.10 to 30 | 0.05 to 20 | 0.1 to 10 | 0.3 | 2.3 |
| Optional additives | to 100 | to 100 | to 100 | to 100 | to 100 |
| | Formula 156 | Formula 157 | Formula 158 | Formula 159 | Formula 160 |
| Polar Solvent a1) | 80 to 99 | 90 to 98 | 92 to 97 | 95.8 | 94.4 |
| Polyol | 0.10 to 20 | 0.05 to 10 | 0.1 to 50. | 0.2 | 0.1 |
| Optional additives | to 100 | to 100 | to 100 | to 100 | to 100 |

| | Formula 161 | Formula 162 | Formula 163 | Formula 164 | Formula 165 |
|---|---|---|---|---|---|
| Polar Solvent a1) | 80 to 99 | 90 to 98 | 92 to 97 | 95.8 | 94.4 |
| Protein Hydrolysate | 0.10 to 10 | 0.05 to 50. | 0.1 to 30. | 0.1 | 0.2 |
| Optional additives | to 100 | to 100 | to 100 | to 100 | to 100 |

| | Formula 166 | Formula 167 | Formula 168 | Formula 169 | Formula 170 |
|---|---|---|---|---|---|
| Polar Solvent a1) | 80 to 99 | 90 to 98 | 92 to 97 | 95.8 | 94.4 |
| Wax | 0.10 to 10 | 0.05 to 50. | 0.1 to 30. | 10. | 1.3 |
| Optional additives | to 100 | to 100 | to 100 | to 100 | to 100 |

Aside from the previously described components a1) to a3), particularly preferred cosmetic preparations contain only slight amounts of further active ingredients and additives. Cosmetic preparations, wherein the proportion by weight of the components a1), a2) to the total weight of the cosmetic preparation is at least about 86% by weight, preferably at least about 90% by weight and particularly at least about 94% by weight, are particularly preferred because they are easily produced and have a good cosmetic effect.

As stated above, the inventive cosmetic preparations a) are particularly suitable for being applied by means of a device for flash evaporation. A further object of the present application therefore is the use of a cosmetic preparation a), containing, based on its total weight,
a1) from about 70 to about 90% by weight of polar solvent;
a2) at least about 0.1% by weight of care component;
as process material in a device for flash evaporation.

The subject matter of the present application moreover is the use of an inventive product for acting upon keratin-containing fibers, especially on human hair, with a cosmetic preparation a) or for the temporary deformation of keratin-containing fibers, especially of human hair.

A method for the temporary deformation of keratin-containing fibers, especially of human hair, for which a device for flash evaporation acts upon the keratin-containing fibers with a cosmetic preparation a) containing, based on its total weight
a1) from about 70 to about 99% by weight of polar solvent;
a2) at least about 0.1% by weight of care component;
is a further subject matter as contemplated herein. The cosmetic preparation a) is converted with the device for flash evaporation preferably into a spray mist, which subsequently acts on the keratin-containing fibers.

In order to achieve a sufficient spraying effect, the cosmetic preparation a) preferably is heated to temperatures above the boiling point of the polar solvent or solvent mixture, contained in the cosmetic preparation a).

If the polar solvent is water or a solvent mixture containing more than about 50% by weight of water (based on the total weight of the solvent mixture), the cosmetic preparation preferably is heated to temperatures above 100° C., preferably to temperatures of from 100° to about 240° C. and particularly to temperatures of from about 140° to about 160° C.

The pressure, achieved by heating the cosmetic preparation a) in the cases in which the polar solvent is water or a solvent mixture containing more than about 50% by weight of water (based on the total weight of the solvent mixture, preferably is between about 1.1 and about 8 bar and particularly between about 1.2 and about 4 bar.

A preferred subject matter of the application is a method for changing the color of keratin-containing fibers, especially human hair, for which the keratin -containing fibers are acted upon by means of a device for flash evaporation with a cosmetic preparation a), which contains, based on its total weight,
a1) from about 70 to about 99% by weight of polar solvent;
a2) at least about 0.1% by weight of care component;
wherein
   from a reservoir, in the interior of which there is a pressure, which corresponds to atmospheric pressure, a partial amount of the cosmetic preparation a), which is in this reservoir, is transferred into a container b1);
   subsequently the access between the reservoir and the container b1) is interrupted by a flow-regulating component, by means of which the flow of the cosmetic preparation a) from the reservoir into the container b1), which can be interrupted, is interrupted;
   subsequently, the cosmetic preparation a) in the container b1), which is closed off from the surroundings, is heated by means of a heater, so that the pressure in the interior of the container b1) rises to values above atmospheric pressure, preferably to values between about 1.1 and about 8 bar and particularly to values between about 1.2 and about 4 bar;
   subsequently, the container b1), which is under a pressure above that of the surroundings, is opened in such a manner, that at least a partial amount, preferably at least about 50% by weight, especially of at least about 80% by weight and particularly at least about 90% of the weight of the cosmetic preparation under pressure in the container b1) escapes into the surroundings with a reduction in the pressure existing in the container b1) at the time that the latter is opened.

The pressure on the cosmetic preparation a) is relieved into the surroundings preferably with the formation of a spray mist of the cosmetic preparation a).

The cosmetic preparation a), the pressure on which is relieved as it emerges from the container b1), preferably is applied on keratinic fibers, especially on human hair.

Methods, in the course of which the cosmetic preparation, as the pressure on it is relieved as it leaves the container b1) and before it impacts on the keratinic fibers, is passed through a nozzle, are particularly preferred.

With respect to further preferred embodiments of the inventive uses and of the inventive method, that, which has been said in connection with the inventive cosmetic preparations a) and the device, applies mutatis mutandis to the flash evaporation b).

The inventive materials, uses and methods and some of their preferred embodiments are exemplified by the following points:
1. Cosmetic product, comprising
   a) a cosmetic preparation containing, based on its total weight,
      a1) from about 70 to about 99% by weight of a polar solvent, wherein
      a2) at least about 0.1% by weight of care component;
   b) a device for the flash evaporation of the cosmetic preparation a).
2. The cosmetic product of point 1, wherein the device for the flash evaporation comprises a container b1) and a heater b2) and is configured in such a way that
   the cosmetic preparation a) can be accommodated in the interior of the container b1)
   the interior of the container b1), which is at least partially filled with the cosmetic preparation a), can be closed off,
   the cosmetic preparation a) in the closed interior of the container b1) can be heated with the heater b2) while the pressure increases,
   the pressure on the heated cosmetic preparation a) from the interior of the container b1) can be lowered by being relieved into the surroundings.
3. The cosmetic product of one of the preceding points, wherein the proportion by weight of the polar solvent a1) is from about 80 to about 98% by weight, preferably from about 90 to about 98% and particularly from about 92 to about 97% by weight of the total weight of the cosmetic preparation a).
4. The cosmetic product of one of the preceding points, wherein the polar solvent a1) has a boiling point (20° C., 1013 mbar) of between about 50° and about 110° C. and, preferably, of between about 70° and about 105° C.
5. The cosmetic product of one of the preceding points, wherein a polar solvent a1) is selected from the group comprising water and ethanol.
6. The cosmetic product of one of the preceding points, wherein the ratio by weight of water and ethanol to the total weight of the polar solvent a1) preferably is at least about 80% by weight, especially at least about 85% by weight, particularly at least about 90% by weight and, more particularly, at least about 95% by weight.
7. The cosmetic product of one of the preceding points, wherein the proportion by weight of water constitutes more than about 80% by weight, preferably more than about 85% by weight and particularly more than about 90% of the total weight of the polar solvent a1).
8. The cosmetic product of one of the preceding claims, wherein the polar solvent a1) of water and ethanol comprises the ratio by weight of water to ethanol is from about 50:1 to about 1:1, preferably from about 40:1 to about 2:1 and particularly from about 25:1 to about 5:1.
9. The cosmetic product of one of the preceding points, wherein the proportion by weight of the care component a2) is from about 0.2 to about 18% by weight and preferably from about 0.5 to about 12% and especially from about 1.0 to about 10% by weight of the total weight of the cosmetic preparation a).
10. The cosmetic product of one of the preceding points, wherein the care component a2) is selected from the group of
   i) cationic polymers;
   ii) cationic surfactants;
   iii) silicones, especially from the group of
      alkoxylated dimethicones, preferably from the group of ethoxylated/propoxylated dimethicones;
      aminofunctional silicones;
   iv) polyols;
   v) protein hydrolysates, especially from the group of hydrolysates of keratin, silk protein and wheat protein;
   vi) waxes
11. The cosmetic product of one of the preceding points, wherein the care component a2) is selected from the group of cationic care components, especially from the group of cationic polymers, especially from the group of cationic cellulose derivatives.
12. The cosmetic product of one of the preceding points, wherein the care component a2) is selected from the group of cationic surfactants, especially from the group of quaternary compounds, the ester esterquats and the amidoamines, preferably from the group of quaternary ammonium compounds, preferably from the group of quaternary ammonium compounds, especially from the group of (C₁₂ to C18) alkyl trimethylammonium salts.
13. The cosmetic product of one of the preceding points, wherein the care components a2) are selected from the group of silicones, especially from the group of
   alkoxylated dimethicones, preferably from the group of ethoxylated/propoxylated dimethicones;
   aminofunctional silicones;
14. The cosmetic product of one of the preceding points, wherein the care component a2) is selected from the group of polyols, especially from the group comprising glycerin, sorbitol and panthenol (different from a1).
15. The cosmetic product of one of the preceding points, wherein the care component a2) is selected from the group of protein hydrolysates, especially from the group of hydrolysates of keratin, silk protein and wheat protein.
16. The cosmetic product of one of the preceding points, wherein the care component a2) is selected from the group of waxes, preferably waxes with melting points above about 40° C., especially above about 50° C. and particularly between about 50° and about 90° C.
17. The cosmetic product of one of the preceding points, wherein the cosmetic preparation comprises preferably three and particularly at least for care components a2), which are different from one another.
18. The cosmetic product of point 17, wherein the cosmetic preparation, as care component a2), comprises a cationic polymer and at least one cationic surfactant.
19. The cosmetic product of point 17, wherein the cosmetic preparation, as care component a2), comprises a cationic polymer and at least one cationic surfactant.
20. The cosmetic product of point 17, wherein the cosmetic preparation, as care component a2,) comprises a cationic polymer and at least one protein hydrolysate.
21. The cosmetic product of point 17, wherein the cosmetic preparation, as care component a2), comprises at least one cationic polymer and at least one protein hydrolysate.
22. The cosmetic product of point 17, wherein the cosmetic preparation, as care component a2), comprises at least one silicone and at least one protein hydrolysate.
23. The cosmetic product of one of the preceding points, wherein the cosmetic preparation a), based on its total weight, includes at least about 86% by weight, preferably of at least about 90% by weight and particularly of at least about 94% by weight of the components a1) and a2).
24. Use of a cosmetic preparation a) containing, based on its total weight,
   a) a cosmetic preparation containing, based on its total weight,
      a1) from about 70 to about 90% by weight of polar solvent;
      a2) at least about 0.1% by weight of care components;
   as process materials in a device for flash evaporation.
25. The use of a product of one of the points 1 to 23 for impacting on keratin-containing fibers, especially human hair, with a cosmetic preparation a).
26. The use of a product of one of the points 1 to 23 for the care of keratin-containing fibers, especially human hair.
27. A method for the care of keratin-containing fibers, especially of human hair, for which the keratin-containing fibers are acted upon with a device for the flash evaporation of cosmetic preparation a) containing, based on its total weight
   a1) from about 70 to about 99% by weight of polar solvent;
   a2) at least about 0.1% by weight of care components.
28. The method of point 27, wherein
   from a reservoir, in the interior of which there is a pressure, which corresponds to atmospheric pressure, a partial amount of the cosmetic preparation a), which is in this reservoir, is transferred into a container b1);
   subsequently the access between the reservoir and the container b1) is interrupted by a flow-regulating component, by means of which the flow of the cosmetic preparation a) from the reservoir into the container b1), which can be interrupted, is interrupted;
   subsequently, the cosmetic preparation a), closed off from the surroundings in the container b1), is heated by means of a heater, so that the pressure in the interior of the container b1) is heated to values above the surrounding pressure, preferably to values between about 1.1 and about 8 bar, especially to values between about 1.2 and about 4 bar, subsequently the container b1), which is under a pressure above atmospheric pressure, is opened in a way, which enables at least a partial amount, preferably at least about 50% by weight, particularly at least about 80% by weight and more particularly at least about 90% of the weight of the cosmetic preparation in the container b1) to emerge from the container b1) into the atmosphere with a reduction in pressure existing in the container b1 at the time that the latter is opened.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic product, comprising:
a) a cosmetic preparation comprising, based on its total weight,
a1) from about 70 to about 99% by weight of a polar solvent,
a2) at least about 0.1% by weight of care components;
b) a device for the flash evaporation of the cosmetic preparation a).

2. The cosmetic product of claim 1, wherein the device b) for the flash evaporation comprises a container b1) and a heater b2) and is configured in such a way that
the cosmetic preparation a) can be accommodated in the interior of the container b1),
the interior of the container b1), which is at least partially filled with the cosmetic preparation a), can be closed off,
the cosmetic preparation a) in the closed interior of the container b1) can be heated with the heater b2) while the pressure increases,
the pressure on the heated cosmetic preparation a) from the interior of the container b1) can be lowered by being relieved into the surroundings.

3. The cosmetic product of claim 1, wherein the proportion by weight of the polar solvent a1) is from about 80 to about 99% by weight based on the total weight of the cosmetic preparation a).

4. The cosmetic product of claim 1, wherein the polar solvent a1) is selected from the group of water, ethanol, or a combination thereof.

5. The cosmetic product of claim 1, wherein the proportion by weight of care component a2) is from about 0.2 to about 18% by weight, based on the total weight of the cosmetic preparation a).

6. The cosmetic product of claim 1, wherein the care component a2) is selected from the group of
i) cationic care components
ii) silicones
iii) polyols;
iv) protein hydrolysates,
v) waxes,
or combinations thereof.

7. The cosmetic product of claim 1 wherein the cosmetic preparation comprises at least two care components a2), which are different from one another.

8. (canceled)

9. (canceled)

10. (canceled)

11. A method for the care of keratin-containing fibers, wherein the method comprises:
acting upon the keratin-containing fibers arc acted upon with a device for the flash evaporation of a cosmetic preparation a), wherein the cosmetic preparation comprises, based on its total weight:
a1) from about 70 to about 99% by weight of a polar solvent
a2) at least about 0.1% by weight of care components.

12. The method of claim 11, further comprising:
transferring a partial amount of the cosmetic preparation a) from a reservoir, in the interior of which there is a pressure, which corresponds to atmospheric pressure, into a container_b1);
subsequent to transferring the partial amount of the partial amount of the cosmetic parathion a), interrupting the access between the reservoir and the container b1) by a flow-regulating component,

13. The cosmetic product of claim 1, wherein the proportion by weight of the polar solvent a1) is from about 92 to about 97% by weight based on the total weight of the cosmetic preparation a).

14. The cosmetic product of claim 1, wherein the polar solvent a1) includes water and ethanol in a ratio by weight of water to ethanol of from about 50:1 to about 1:1.

15. The cosmetic product of claim 1, wherein the proportion by weight of care component a2) is from about 1.0 to about 10% by weight based on the total weight of the cosmetic preparation a).

16. The cosmetic product of claim 1, wherein the care component a2) includes cationic care components chosen from the group of cationic polymers and cationic surfactants.

17. The cosmetic product of claim 1, wherein the care component a2) includes silicones chosen from the group of
alkoxylated dimethicones, preferably from the group of ethoxylated/propoxylated dimethicones; or
aminofunctional silicones;

18. The cosmetic product of claim 1, wherein the care component a2) includes protein hydrolysates chosen from the group of hydrolysates of keratin, silk protein, wheat protein, or combinations thereof.

19. The cosmetic product of claim 1, wherein the cosmetic preparation, as care component a2), comprises at least one protein hydrolysate and at least one additional care component chosen from a cationic polymer, a cationic surfactant, or a silicon.

20. The cosmetic product of claim 1, wherein the cosmetic preparation a), based on its total weight, includes at least about 94% by weight of the components a1) and a2).

21. The method of claim 12, further comprising:
subsequent to interrupting the access between the reservoir and the container b1) by the flow-regulating component, heating the cosmetic preparation a) in the container b1), which is closed off from the surroundings, using a heater, wherein the pressure in the interior of the container b1) rises to values above atmospheric pressure.

22. The method of claim 21, further comprising:
subsequent to heating the cosmetic preparation a), opening the container b1), which is under a pressure above that of the surroundings, in such a manner, that at least a partial amount of the cosmetic preparation under pressure in the container b1) escapes into the surroundings with a reduction in the pressure existing in the container b1) at the time that the container b1) is opened.

23. A cosmetic product, comprising:
a) a cosmetic preparation comprising, based on its total weight,
a1) from about 70 to about 99% by weight of a polar solvent,
a2) from about 1.0 to about 10% by weight of care components, wherein the care components a2) are selected from the group of:
i) cationic care components;
ii) silicones;
iii) polyols;
iv) protein hydrolysates,
v) waxes,
or combinations thereof;
b) a device for the flash evaporation of the cosmetic preparation a), wherein the device b) comprises a container b1) and a heater b2) and is configured in such a way that:
the cosmetic preparation a) can be accommodated in the interior of the container b 1),
the interior of the container b1), which is at least partially filled with the cosmetic preparation a), can be closed off,
the cosmetic preparation a) in the closed interior of the container b1) can be heated with the heater b2) while the pressure increases, and
the pressure on the heated cosmetic preparation a) from the interior of the container b1) can be lowered by being relieved into the surroundings.
